(19) United States
(12) Patent Application Publication
(10) Pub. No.: US 2019/0216952 A1
(43) Pub. Date: Jul. 18, 2019

(54) Method for substantiating validating products for environmental safety

(71) Applicant: Joseph Charles DiNardo, Vesuvius, VA (US)

(72) Inventor: Joseph Charles DiNardo, Vesuvius, VA (US)

(21) Appl. No.: 15/870,924

(22) Filed: Jan. 13, 2018

(51) Int. Cl. A61K 49/00

(52) U.S. Cl. CPC A61K 49/0004

(57) ABSTRACT

A method for characterizing the environmental toxicological potential in multiple aquatic species of any chemical or biochemical, packaging material, personal care, household or industrial product, prescription or over-the-counter drug product used in humans, animals and/or structures that can enter fresh, brackish and/or salt water environments including private and public pools, and which may endanger human and/or animal life.

In 2008, Danovaro et al⁶ demonstrated that the ultraviolet (UV) chemical filters Oxybenzone (also know as Benzophenone-3), Octinoxate (also know as Octyl Methoxycinnamate), and 4-Methylbenzylidene Camphor (also known as 4-MBC), along with the preservative Butylparaben caused coral bleaching/death. In addition, these researchers estimated that 4,000 to 6,000 tons of sunscreen products wash off people and enter into in areas harboring coral reefs, thereby directly threatening 10% of the world reefs from this source alone. Additional research published by Downs et al⁷ confirm the toxicity of Oxybenzone (Benzophenone-3) in coral and, furthermore, identified concentrations of this ingredient at toxic levels in various bays and open waters in both the Virgin Islands and the Hawaiian Islands. Tsui et al⁸ identified twelve widely used organic UV chemical filters in surface water that can be harmful to coral. The study collected surface water in eight cities across four countries (China, the United States, Japan, and Thailand) and the North American Arctic. Results indicated a significant number of harmful compounds were detected: Hong Kong (12), Tokyo (9), Bangkok (9), New York (8), Los Angeles (8), Arctic (6), Shantou (5) and Chaozhou (5), and generally increased with population density. Moreover, the study identified these compounds in the North American Arctic suggesting, in part, that long-range oceanic transport of toxic chemicals can cause negative impacts on marine environments in sparsely populated areas, implying the potential for damage even in marine protected areas.

The research reported by Danovaro, combined with the lack of uniform and validated testing of sunscreen products for aquatic toxicity, has produced an unfortunate outcome in the marketplace whereby sunscreens are being promoted as “non-toxic to sea life”, “eco-safe”, “reef friendly”, etc., so long as the product does not contain the specific coral-toxic chemicals identified in the report. This line of reasoning has been further extrapolated to claim that if a sunscreen product contains Zinc Oxide and/or Titanium Dioxide as active ingredient(s) in a formulation free of nano-size particles, then that product will not harm aquatic life and is safe for use in all aquatic settings regardless of the complexity of the formula. Unfortunately, this is a very oversimplified and misleading paradigm that fails to reflect the true toxicity potential of a complex mixture of chemicals used in sunscreen products and how that can and should be determined. Similarly misleading is the belief that if you test an ingredient or product in a single species (e.g., coral) and it does not produce a toxic effect, that it must be safe for all marine life. Of course, this is also inaccurate as each species demonstrates different tolerances to different ingredients and/or products.

### SUMMARY OF THE INVENTION

This patent proposes using a tiered approach for evaluating product safety in marine life, whereby a product is tested in at least 4 different species: Preferably, Embryonic/Larvae of the Phylum Athropoda (preferably in the shrimp family), Phylum Echinoderm (preferably in the sea urchin family), Phylum Chordata (preferably in the clown fish family) and Phylum Cnidaria specifically in the class of Anthozoa (preferably either in a coral fragment or coral cell or coral planula). Additional species maybe tested from various aquatic Phyla if a better representation of a specific environment (fresh, brackish or salt waters) is desirable.

As a matter of practicality, the testing paradigm presented in this document is not envisioned as being inclusive of all marine species; however, the implementation of a uniform and scientifically valid method of effectively testing the toxic potential of substances on harbinger aquatic species is intended to provide an important advance in assessing the likely impact of those substances on the marine environment. While the data presented herewith is limited to a sunscreen and after sun moisturizer, it is important to recognize that many of the same ingredients are used in numerous product types/categories and, therefore are not mutually exclusive to a range of household and personal care products. Thus, the proposed testing paradigm allows for a critical evaluation of the potential impact that a substance may have once it enters water—be it oceans, seas, lakes, streams, ponds, water treatment plants, etc.—thereby affording the opportunity to minimize negative impact on the environment though the avoidance of toxic product formulations.

As noted above, it is essential when determining the Aquatic Toxicity Potential (ATP) of any substance that testing be carried out in at least four different species since different organisms respond differently to a substance and/or a complex mixture. Finally, the methodology described herein provides a means to convey essential and validated information to consumers via an “Eco-friendly” seal that identifies a product as having been tested and, based on the best currently available science, found to be acceptable to aquatic life.

### BRIEF DESCRIPTION OF THE DRAWINGS

FIG. 1: shows a graph depicting the mortality rate (MR) as a percentile after 48 hours of exposure of a SPF 25 Sunscreen in 4 different species at a concentration of 500 ppb.

FIG. 2: shows a graph depicting mortality rate (MR) as a percentile after 48 hours of an After Sun Moisturizer in 4 different species at a concentration of 500 ppb.

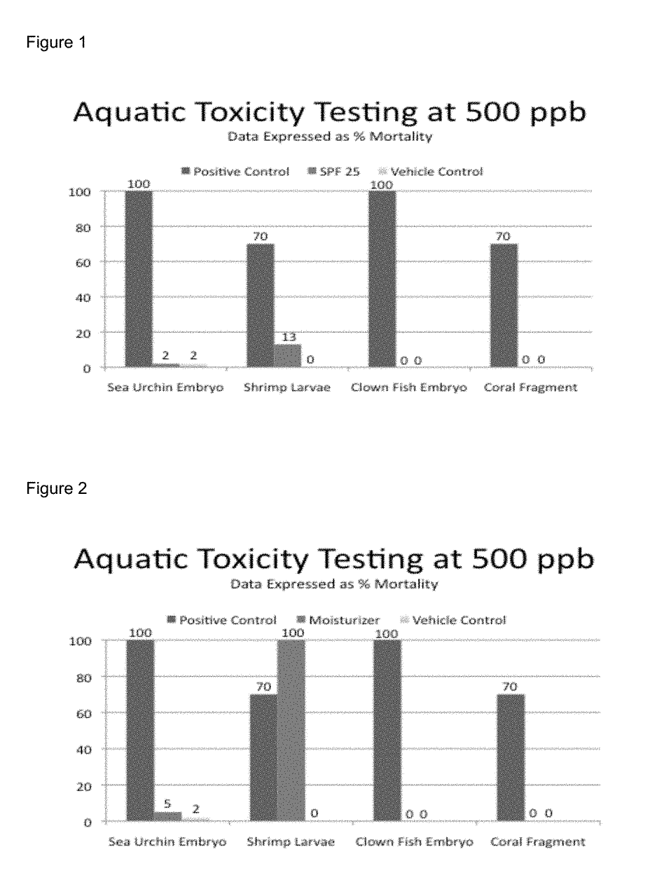

Four different species of aquatic life were tested to determine the toxicity of two experimental sun care products designed to be safe for marine life. The SPF 25 broad-spectrum water-resistant formula contained 16 ingredients including 11.4% of a non-nano particle size Zinc Oxide (active ingredient) and the After Sun Moisturizer contained 9 basic ingredients; neither formulation was preserved with parabens. The Positive Control was a commercially available sunscreen which contained 43 ingredients including 2% Avobenzone, 5.5% Homosalate, 4.0% Octisalate and 4.0% Octocrylene as sunscreen actives, and also contained parabens as part of the preservative system. The Vehicle Control was simply seawater.

The vehicle control produced little to no toxicity in all of the species tested, while the positive control produced 100%, 70%, 100% and 70% MR in embryo/larvae of sea urchin, shrimp, clown fish and coral fragment, respectively. In contrast, the experimental SPF 25 broad-spectrum water-resistant formula produced a 2%, 13%, 0%, 0% MR and the After Sun Moisturizer a 5%, 100%, 0%, 0% MR in embryo/larvae sea urchin, shrimp, clown fish and coral fragment, respectively. A MR above 30% in any species was considered a toxic effect and, therefore, based on this criteria only the experimental SPF 25 broad-spectrum water-resistant formula and the vehicle control could be considered safe for marine life.

The specific example reported in this research clearly demonstrates the need for product testing in multiple species prior to being sold as safe to marine life. Both the experimental SPF 25 broad-spectrum water-resistant formula and the After Sun Moisturizer would have been considered safe for marine life based on the inspection of the ingredient label and current marketing practice in sun care products (i.e., contains few ingredient, does not contain parabens and/or uses either Zinc Oxide and/or Titanium Dioxide actives). However, in the course of testing in multiple species, the After Sun Moisturizer produced a MR of 100% in shrimp embryo/larvae, which was an unexpected result. Upon further research of the literature and evaluation of the formula, Phenoxyethanol (used to preserve the product), was found to have originally been used as an insecticide/insect repellant and was considered toxic at low concentrations to invertebrates like shrimp⁹. Therefore, if the product were only tested in sea urchins, clown fish or coral the product would have been mislabeled as safe for marine life. Moreover, the testing methodology was successful in flagging a previously unexpected toxic ingredient, thereby allowing for re-formulation and re-testing prior to any commercialization.

### DETAILED DESCRIPTION

The present invention evaluates two experimental personal care products in four aquatic toxicity assays. Products tested: Eco-friendly SPF 25 broad-spectrum water-resistant sunscreen and Eco-friendly After Sun Moisturizer. The four tests used to assess the aquatic toxicity potential (ATP) of each product are identified with references below.

Test 1: Athropoda phylum: Shrimp Embryonic/Larvae Acute Toxicity Assay conducted using a modification of the EPA Methods for Assessing the Toxicity of Sediment-associated Contaminants with Estuarine and Marine Amphipods¹.

Test 2: Echinoderm phylum: Sea Urchin Embryonic/Larvae Acute Toxicity Assay conducted using a modification of the method described in Laboratory Toxicity Testing for the Meso-American Reef—Chapter 4².

Test 3: Chordata phylum: Fish Embryonic/Larvae Acute Toxicity Assay conducted using a modification of the OECD Guidelines For The Testing of Chemicals No. 236 Fish Embryo Acute Toxicity Adopted July 2013³.

Test 4: Cnidaria phylum: Coral Fragment, Coral Cell or Coral Planula Larvae Toxicity Assay conducted using a modification in Laboratory Toxicity Testing for the Meso-American Reef—Chapters 1-3².

Modification: 48 h exposure time with lethality as the final endpoint.

The methods noted above are used to evaluate the ATP of species living in salt water. Depending on the specific species/environment being evaluated for ecological impact fresh and/or brackish water species can be substituted as long as the vehicle used to dilute a test substance and the vehicle control matches the environment being studied.

While the invention has been described in connection with individual embodiments, it is not intended to limit the scope of the invention to the particular form set forth; rather, it is intended to cover all such alternatives, modifications and equivalents as may be included to evaluate toxicity to an aquatic environment.

What is claimed is:

1. A method for characterizing the environmental toxicological potential in multiple aquatic embryonic/larvae species to a test substance (an individual chemical, biochemical, package or packaging resin or a finished product), whereby the method is comprised of: obtaining a first result in the first species tested by which the first result is indicative of the aqua toxicity potential (ATP), relative to survival of the species being exposed for 48 hours to the test substance with a mortality rate (MR) less than or equal to 30%. If the first test result produces an MR equal to or below 30%, the test substance can be tested in the second species, under the same conductions, to evaluate the ATP. If the second test result produces an MR equal to or below 30%, the test substance can be tested in the third species, under the same conductions, to evaluate the ATP. If the third test result produces an MR equal to or below 30% the test substance can be tested in the fourth species, under the same conductions, to evaluate the ATP. If the test substance is observed to produce an ATP equal to or less than 30% in all four species, it can be considered Eco-friendly (i.e., not environmentally harmful) and a “seal of approval” given that can be placed on the product package that clearly designates this status to the consumer.

2. The method as recited in claim 1 wherein the first ATP is obtained in the Phylum Athropoda, preferably in shrimp embryonic/larvae, being exposed to the environmental stressor (test substance).

3. The method as recited in claim 1 wherein the second ATP is obtained in the Phylum Echinoderm, preferably in sea urchin embryonic/larvae, being exposed to the environmental stressor (test substance).

4. The method as recited in claim 1 wherein the third ATP is obtained in the Phylum Chordata, preferably in clown fish embryonic/larvae, being exposed to the environmental stressor (test substance).

5. The method as recited in claim 1 wherein the forth ATP is obtained in the Phylum Cnidaria, preferably in coral fragment, coral cell or coral embryonic/larvae, being exposed to the environmental stressor (test substance).

6. The method as recited in claim 1 wherein the first score is based on the ATP in the first species resulting in a MR of less than or equal to 30% prior to being testing in the second species.

7. The method as recited in claim 6 wherein the second score is based on the ATP in the second species resulting in a MR of less than or equal to 30% prior to being testing in the third species.

8. The method as recited in claim 7 wherein the third score is based on the ATP in the third species resulting in a MR of less than or equal to 30% prior to being testing in the forth species.

9. The method as recited in claim 8 wherein the forth score is based on the ATP in the forth species resulting in a MR of less than or equal to 30% prior to being considered Eco-friendly.

10. The method as recited in claim 1 wherein a vehicle control (fresh, brackish or salt water neat depending on the species being tested) must be tested concurrently in each ATP evaluation and species tested.

11. The method as recited in claim 1 wherein the test substance is evaluated at a concentration between 1 parts per million (ppm) and 1 parts per trillion (ppt), preferably at 500 parts per billion (pph); depending upon solubility, exposure levels in the ecosystem being study and or species being evaluated.

12. The method as recited in claim 1 wherein the test substance being evaluated is not restricted to being tested in the preferable species noted in claims 2, 3, 4 and 5, but rather tested in four appropriate species based on the aquatic environment (fresh, brackish and/or salt water) being studied.
